(19) United States
(12) Patent Application Publication
(10) Pub. No.: US 2017/0241990 A1
(43) Pub. Date: Aug. 24, 2017

(54) PERICYTE ASSAY FOR TRANSENDOTHELIAL MIGRATION

(71) Applicant: Case Western Reserve University, Cleveland, OH (US)

(72) Inventors: Diego Correa, Cleveland, OH (US); Arnold I. Caplan, Cleveland, OH (US)

(21) Appl. No.: 15/327,332

(22) PCT Filed: Jul. 20, 2015
(86) PCT No.: PCT/US15/41061

§ 371 Date: Jan. 18, 2017

Related U.S. Application Data
(60) Provisional application No. 62/026,081, filed on Jul. 18, 2014.

(51) Int. Cl. G01N 33/50

(52) U.S. Cl. CPC G01N 33/5088; G01N 33/5032; G01N 33/5011; G01N 2500/10

(57) ABSTRACT

A test animal for identifying blood vessel transendothelial migration-affecting factors or agents is described. The test animal comprises an implanted tissue mimicking model with a composite vascular structure comprising pericytes on test animal vasculature. The test animal can be used in a method of identifying or evaluating a blood vessel transendothelial migration-affecting agent that includes administering the agent to a test animal and determining the effect of the agent on the level of blood vessel transendothelial migration into or out of the tissue model. The test animal can also be used in a method of identifying or characterizing a metastasis-facilitating factor that includes administering or inducing circulating tumor cells in a test animal in which one or more factors in the pericyte have been silenced, and determining if the level of tumor cell migration into the tissue model has increased or decreased.

### CONTINUING APPLICATION DATA

This application claims the benefit of U.S. Provisional Application Ser. No. 62/026,081 filed Jul. 18, 2014, which is incorporated by reference herein.

### GOVERNMENT FUNDING

This work was supported, at least in part, by Grant No. RO1 CA163562-01A1 from the National Cancer Institute of the National Institutes of Health. The United States government has certain rights in this invention.

### BACKGROUND

Mesenchymal Stem Cells (MSC) have long been described as cellular progenitors of mesenchymal lineages including bone, cartilage, fat, muscle and other connective tissues. In addition, a secretory capacity has been identified with both immunomodulatory and trophic activities, exerted at sites of injury where they interact with other local cellular components. The recent identification of the MSC niche in the perivascular space as pericytes has opened the possibility that MSC may have additional roles directly controlling tissue homeostasis from their cardinal abluminal location. Caplan, A I., Cell Stem Cell 2008; 3(3), 229-230.

Metastasis is a leading prognostic indicator for cancer survival and a major contributor to cancer mortality. The skeleton and liver are preferred organs for cancer dissemination in various malignancies including malignant melanoma. Although the preponderance of distant metastases implies a selective advantage for arriving disseminating cells, recent studies have determined that the rates of tumor growth, invasion, and metastasis are in fact independent pathological traits governed by different sets of genes. Nguyen D X, Massague J, Nat Rev Genet 2007; 8(5), 341-352. Nevertheless, they share a common feature, namely their dependence on the vasculature that provides access to oxygen and nutrients, as well as a route for cancer cell dissemination. Current cancer therapies are designed to alter not only specific biological functions in cancer cells, but also to target components of the tumor microenvironment (TME)/stroma, especially the vasculature.

Pericytes are a specialized cell type that function abluminally covering and stabilizing blood vessels following their recruitment to forming vessels as progenitor cells via PDGF-B/PDGFRB signaling. Armulik et al., Circ Res 2005; 97(6), 512-523. Pericyte involvement in primary tumors growth constitutes a novel therapeutic target based on compelling evidence showing superior reduction in size when targeted in parallel with endothelial cells (ECs). Bergers et al., J Clin Invest 2003; 111(9), 1287-1295. However, pericyte coverage of the vasculature seems to differentially regulate tumor growth and metastatic potential, as intravasation of cancer cells is increased in primary tumors grown in mice with a genetically-determined deficient pericyte coverage. Xian et al., J Clin Invest 2006; 116(3), 642-651. These findings have led to the appreciation of pericytes in primary tumors as gatekeepers for cancer dissemination. Gerhardt H, Semb H., J Mol Med 2008; 86(2), 135-144. In parallel, in response to tumor signals, BM-derived progenitor cells of mesenchymal origin (as MSC) are recruited to the tumor stroma localizing in perivascular sites and helping to assemble a supporting vascular network critical for tumor growth. Bergfeld S A, DeClerck Y A., Cancer metastasis reviews 2010; 29(2), 249-261. Taken together, BM-derived MSC (BM-MSC) appear to play a critical role during primary tumor formation, growth and subsequent invasive potential.

In contrast, the role of pericytes at the target organ microenvironment during cancer cell extravasation is not known. Recently, data suggest that cellular and molecular stromal elements in the BM are related to the establishment and progression of skeletal metastasis. For example, blocking PDGFB signaling with a multi-target tyrosine kinase inhibitor (Sunitinib) impairs bone invasion of circulating osteotropic lung cancer cell lines due to altered tumor cell-BM stroma interactions. Catena et al., Cancer Research 2011; 71(1), 164-174. In addition, it has been established that invading cancer cells physically associate with mesenchymal derived cells in the BM stroma affecting various biological activities of engrafted cancer cells, including dormancy/quiescence, resistance to chemotherapy and metastatic growth. Corcoran et al., PLoS One 2008; 3(6), e2563. However, the specific identity of the cellular and molecular elements, as well as the precise location where the sequence of events occur during extravasation is still not well understood.

The mechanisms governing skeletal metastasis involve comparable details to those used by hematopoietic stem cells (HSC) entering the BM. This “homing” behavior relies on the existence of a specific physical niche within the BM where other cellular players, including MSC, favor the constant trafficking of such progenitors across the sinusoidal wall. Shiozawa et al showed that invading osteotropic prostate cancer cells enter the HSC niche competing with resident cells and thus establishing physical anchors for further growth inside the BM. Shiozawa et al., The Journal of clinical investigation, 2011; 121(4), 1298-1312.

It is well known that current animal models for cancer research are inadequate, and that new animal models are a key to improving cancer therapy. Wagner, Kay-Uwe, Breast Cancer Res 2004, 6:31-38. Given the importance of transendothelial migration in cancer metastasis and other important biological processes, there is a need for animal models that can be used to identify key factors that can serve as targets for agents affecting transendothelial migration.

### SUMMARY

With the notion of BM-MSC as pericytes, a proposal can be constructed centered around the physical interaction of invading cancer cells and resident BM-MSC occurring at the abluminal space of BM sinusoids as a determinant step in the initiation and fate of skeletal metastasis. The inventors have shown that altering the physical interaction between vascular components of the target organ microenvironment (ECs and MSC/pericytes) via genetic manipulation of PDGF-B, dramatically impairs the engraftment of intraarterially-delivered MCC, thus reducing the frequency of osteolytic bone metastasis. Through in vitro and in vivo approaches, including a humanized assay in which fully functional extraskeletal bones are engineered with human MSC (hMSC), the essential molecular players and mechanisms involved in the extravasation of circulating MCC to the BM were established, that become disrupted in the absence of sinusoidal MSC/pericytes. In parallel, they observed that the situation in the BM is replicated in the liver exclusively, whereas no invasion by melanoma was seen in mutant mice. The inventors therefore propose that the presence of MSC as pericytes surrounding BM and liver sinusoids is required for extravasation of MCC, and that the effects of the EC/pericyte dissociation at the metastatic target organ do not mirror its effects during intravasation at the primary tumor.

The inventors show that MSC/pericytes function as sentinels regulating cancer cell dissemination with a differential effect during intravasation at the primary tumor and extravasation at the target organ. The molecular mechanisms, cellular players and locations during the establishment of melanoma metastasis to BM and liver were defined. This provides clinically relevant information for the design of novel therapeutic strategies aimed at reducing engraftment by closing the gate through which metastatic cells transit into the target organ.

### BRIEF DESCRIPTIONS OF THE DRAWINGS

FIG. 1(A,B) provides images showing BM vascular appearance and micro computed tomography (μCT) assessment of invaded bones. (A) CD31 (Pecam) immunostaining of PDGF-B mutant and Het mice (control) BM, showing no signs of blood vessels gross abnormalities such as collapse and hemorrhages. (B) Gross appearance, two-dimensional CT scanning, and tri-dimensional renderings-volumetric and surface of Het (control) and PDGF-B mutant (Mut) mice humeri. Note the marked osteolysis area in the Het humerus (arrow).

FIG. 2(A-C) provides graphs and images of BLI of injected PDGF-B mutant and control mice (Het and WT): (A) Imaging 15 minutes, 5 and 12 days post-injection, showing increased skeletal invasion (limbs and spine) in WT and Het mice compared to PDGF-B mutant mice and the disappearance of a metastatic signal at 12 days in PDGF-B mutant mice (grey circles). (B) Absence in PDGF-B mutant and persistence in Het mice of the spine signal (white circle) after adrenal gland removal (white arrows) confirming the non-spinal origin of the signal in PDGF5-B mutant spine (arrow in A). (C) Quantification of signal (photon flux and area covered by tumors) showing statistical difference between PDGF-B mutant and Het mice (*=p<0.01). Data are represented as mean±SEM. Representative mice of n=15 (5 per group).

FIG. 3(A,B) provides images of gross inspection of distant melanoma dissemination: (A) Craniofacial and appendicular invasion by MCC exhibiting a significant reduction in PDGF-B mutant mice. All skulls and scapulae in the mutants were clear of metastases or had only few/small foci, which contrasts sharply with the multiple major/multifocal invasion observed in Het mice. Spines in PDGF-B mutant mice were clear of metastasis in 2/5 mice, or harbored only 1-2 small foci restricted to one vertebral segment in the remaining 3 animals. WT and Het mice had multiple multi-segment metastases in all animals. Both distal femur and proximal tibia were compromised bilaterally in all WT and Het controls, while bilateral invasion was observed in only 1 of 5 PDGF-B mutant mice, with remaining 4 mice exhibiting only one compromised leg that was restricted to the proximal tibia. (B) Melanoma invasion to other target organs with comparable results in both genotypes except for liver (reduced in PDGF-B mutant mice). Arrows: adrenal glands. Circle: adrenal gland agenesis. Three representative mice shown of n=15 (5 per group).

FIG. 4(A-C) provides images showing the histology of metastatic bone tumors. (A, B) Metastatic tumors (T) in distal femur (A) and liver (B) are smaller or absent in PDGF-B mutant mice. Bars=200 μm (low magnification) and 10 μm (high magnification). Melanin-producing B16F10 cells (arrows) localize in the abluminal side of BM sinusoids (Sin), extending to the tissue parenchyma (impaired in PDGF-B mutant mice). (C) CD146 immunohistochemistry (IHC) in BM sections from Het mice. Engrafted B16F10 cells (arrows) physically associate with CD146-positive BM-MSC/pericytes (signal) at the perivascular space and inside the parenchyma (circle). Dotted line=boundary between tumor-invaded and tumor-free BM. Bar=10 μm.

FIG. 5(A-C) provides graphs and images showing induced gene silencing in B16F10 and hMSCs. Effective CD146 silencing (˜90%) in B16F10 (A) and hMSCs (˜75%) (B), assessed by immunocytochemistry (ICC) in cultured cells. (C) Sdf-1/CXCL12 effective gene silencing (˜55%) in hMSCs evidenced by qPCR from RNA obtained from cultured cells. *(p<0.05). Bars=200 μm.

FIG. 6(A-C) provides graphs and images showing the invasion of engineered B16F10 MCC: Reduced invasion of CD146-silenced MCC to craniofacial, appendicular structures and liver (compared to NT-shRNA), evaluated by BLI (A) and gross examination (B & C). BLI signal reached statistical significance (*=p<0.01). Data are represented as mean±SEM. Representative mice of n=6 (3 per group).

FIG. 7 provides a schematic representation of humanized extraskeletal ossicle conformation. The schematic representation of the ossicle showing theoretical cell dispositions within the tissue structure before and after silencing induction with IPTG administration in the drinking water. A timeline of the experiment is also shown. Adip=Adipocyte; Sin=Sinusoid; MK=Megakaryocyte.

FIG. 8(A,B) provides images showing the human origin of perivascular cells in ossicles and in vivo silencing of CD146. (A) Immunodetection (IHC) of the MSC marker CD271 (blue signal) with a human specific antibody evidences the donor (human) origin of perivascular cells in the engineered ossicles. (B) Immunodetection (IHC) of CD146 in ossicle sections revealing its presence in perivascular cells (arrows) in control structures, significantly reduced in ossicles made with CD146-silenced hMSCs. Bars=200 μm.

FIG. 9(A-C) provides graphs and images showing the B16F10 MCC invasion to humanized extraskeletal ossicles. (A) MCC invasion of the skeleton (top row BLI) and specific implanted ossicles, evaluated by direct examination after harvesting (middle row) and by BLI (bottom row). The signal from the cubes BLI was quantified (photon flux) giving statistical difference of all groups compared to control (*=p<0.01). Data are represented as mean±SEM. Representative mice and cubes of n=8. (B) Histological analysis (H&E staining) of harvested ossicles shows significant melanoma invasion in structures made with control MSC while significantly reduced or absent with Sdf-1/CXCL12 and CD146-silenced cells. SD cubes exhibit no bone and vasculature formation. Bar=200 μm. (C) Immunolocalization (IHC) of CD146 (arrow) in sections from Control ossicles showing invading MCC physically associated with MSC/pericytes at the perivascular space surrounding sinusoids (Sin), and advancing towards the tissue parenchyma as a cell complex (red circle). Bar=10 μm.

FIG. 10(A,B) provides a cross-sectional representation and images showing the in vitro transendothelial migration (TEM) assay: (A) Schematic representation of the modified TEM. (B—top row) Fluorescence microscopy of DiI-labeled hMSC expressing either non-target (NT) or Sdf-1/CXCL12 silencing vectors (Sdf-1_shRNA) and papillary dermal Fibroblasts seeded at the bottom surface of an 8 μm pore diameter insert membrane. (B—bottom row) Merged bright field and fluorescence microscopy showing B16F10 melanoma cell invasion to the membrane and interaction with seeded cells (Bar=200 μm). Representative pictures from three independent experiments.

FIG. 11(A,B) provides a schematic representation and image showing the in vitro transendothelial migration assay and a result from the assay. (A) DiI-labeled hMSC (red) expressing either nontarget (NT) or Sdf-1/CXCL12 silencing vectors (Sdf-1_shRNA) seeded at the surface of the transwell at varying distances from the insert. (B) No invasion of MCC to the membrane was observed at all distances (one field shown), confirming the lack of migration by B16F10 cells.

FIG. 12(A,B) provides a schematic representation of the proposed model. (A) PDGFR-β-expressing MSCs are recruited as pericytes by endothelial cells (EC)-secreted PDGF-B retained in the heparan sulfate-rich pericellular matrix. In their perivascular location, they create an Sdf-1/CXCL12 gradient across the endothelium (black dots) attracting CXCR4-expressing melanoma cells. Sdf-1/CXCL12 is also expressed in smaller quantities by ECs. Cell-cell interactions mediated by homotypic CD146 binding generates that this cellular complex then enters the BM parenchyma. The absence of MSCs in their normal perivascular niche disrupts these mechanisms.

### DETAILED DESCRIPTION

The methods and techniques described herein are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### Definitions

For clarification in understanding and ease in reference a list of terms used throughout the brief description section and the remainder of the application has been compiled here. Some of the terms are well known throughout the field and are defined here for clarity, while some of the terms are unique to this application and therefore have to be defined for proper understanding of the application.

“A” or “an” means herein one or more than one; at least one. Where the plural form is used herein, it generally includes the singular.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning, as does the word “includes.”

“Effective amount” generally means an amount which provides the desired local or systemic effect, e.g., effective to ameliorate undesirable effects of inflammation, including achieving the specific desired effects described in this application. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intra-arterial, intramuscular, subcutaneous, transdermal, airway (aerosol), pulmonary, nasal, rectal, and topical (including buccal and sublingual) administration.

As used herein, “metastasis” refers to the ability of cells of a cancer (e.g. a primary tumor, or a metastasis tumor) to be transmitted to other locations in the subject (i.e., target organs) and to establish new tumors at such locations.

“Mesenchymal stem cells” or “MSCs” are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

“Stem cell” means an undifferentiated cell that can undergo self-renewal (i.e., progeny with the same multi-differentiation potential) and also produce progeny cells that are committed to a particular differentiation lineage. Within the context of the invention, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

The expression “test animal,” as used herein, includes any non-human animal, preferably belonging to the class of mammals, for example, rodents, and specifically mice. In preferred embodiments of the present invention as described above and below, the test animal is preferably a mouse but may be another mammalian species, for example another rodent, for instance a rat, hamster or a guinea pig, or another species such as a chimpanzee, monkey, pig, rabbit, or a canine or feline, or an ungulate species such as ovine, caprine, equine, bovine, or a non-mammalian animal species.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy. In various embodiments, the symptoms of a disease or disorder are alleviated by at least 5%, at least 10%, at least 20%, at least 30%, at least 40%, or at least 50%.

### Identifying Blood Vessel Transendothelial Migration Affecting Agents

In one aspect, the present invention provides a method of identifying or evaluating a blood vessel transendothelial migration-affecting agent. The method includes administering an agent to a test animal including an implanted tissue mimicking model with a composite vascular structure comprising donor (e.g., human) pericytes on test animal vasculature, and determining the effect of the agent on the level of blood vessel transendothelial migration into or out of the tissue model.

Blood vessels are generally cylindrical conduits that are part of the circulatory system that transports blood throughout the body. An arrangement of blood vessels is referred to as vasculature. Blood vessels include arteries, capillaries, and veins. The space within the blood vessel is referred to as the lumen, while the first layer adjacent to the lumen is the endothelium. The endothelium is a single layer of simple squamous endothelial cells glued by a polysaccharide intercellular matrix. Outside of the endothelium is the basement membrane, which is a thin, fibrous, non-cellular region of tissue that separates the endothelium from the surrounding smooth muscle and connective tissue. Capillaries have a significantly simpler structure, consisting of endothelium surrounded by a basement membrane. Capillaries can be fenestrated (i.e., equipped with diaphragms that regulate the size of the fenestration) or non-fenestrated (i.e., open pore capillaries or sinudoids).

Pericytes are contractile cells that wrap around the endothelial cells of blood vessels throughout the body. Also known as Rouget cells or mural cells, pericytes are embedded in basement membrane where they communicate with endothelial cells of the blood vessels by means of both direct physical contact and paracrine signaling. Pericytes regulate capillary blood flow, the clearance and phagocytosis of cellular debris, and the permeability of the blood vessel to other cells, including circulating cancer cells. A variety of integrin molecules and other factors are involved in communication between pericytes and endothelial cells separated by the basement membrane.

Transendothelial migration refers to the migration of cells through the various layers making up the blood vessel, and in particular the endothelial layer. Transendothelial migration includes extravasation and intravasation. Extravasation refers to migration of a cell (e.g., a circulating tumor cell) from within the blood vessel to outside of the blood vessel (e.g, to an organ or tissue such as bone), while intravasation refers to migration of a cell from outside the blood vessel (e.g., at the site of a primary tumor) to within the blood vessel.

In some embodiments of the invention, the transendothelial migration of a cell is referred to as “facilitated.” Facilitated migration is transendothelial migration in which pericytes play an active role in encouraging movement through the endothelium of the blood vessel. Facilitation works via the creation of both cancer cell-attracting molecular gradients and docking points for cell-cell attachment (both exerted by the facilitators). These mechanisms attract cancer cells close to the endothelium capturing them with further migration outside the blood vessel. For example, carbohydrate-mediated cancer cell adhesion to the vascular endothelium is involved in the metastasis of a wide variety of cancers, including gastric, colorectal, pancreatic, liver, ovary, head and neck, and breast cancers, etc. In some embodiments, blood vessel transendothelial migration is facilitated extravasation, while in other embodiments blood vessel transendothelial migration is facilitated intravasation. In further embodiments, the blood vessel transendothelial migration is the facilitated extravasation of circulating cancer cells (e.g., melanoma cells) into the tissue model (e.g., bone tissue) of the invention.

The method of the invention can be used to identify or evaluate agents capable of affecting blood vessel transendothelial migration in a variety of different contexts, and from a variety of different sources. For example, blood vessel transendothelial migration can occur during normal biological processes such as the immune response (e.g., leukocyte migration) and wound healing. Because blood vessel transendothelial migration includes both extravasation and intravasation, in some embodiments, the effect of the agent on facilitated blood vessel extravasation is determined, whereas in other embodiments the effect of the agent on facilitated blood vessel intravasation is determined. Identifying an agent refers to finding a new agent whose ability to affect blood vessel transendothelial migration was previously unknown, while evaluating an agent refers to characterizing the activity of an agent which was previously shown to affect blood vessel transendothelial migration.

In a preferred embodiment, the invention provides a method for identifying an agent that inhibits the blood vessel transendothelial migration of a metastatic cancer cell. As used herein, the term “an agent that inhibits the migration a metastatic cancer cell” includes an agent that inhibits the blood vessel transendothelial migration of a metastatic cancer cell in a subject, or an agent that is a candidate for further testing. Such inhibitory agents include, e.g., agents that inhibit the metastasis of a cancer cell from a primary tumor, or the further metastasis of a cancer cell from a site of metastasis. Accordingly, in some embodiments, the blood vessel transendothelial migration-affecting agent is an antimetastatic agent, and the blood vessel transendothelial migration is the facilitated transport of circulating cancer cells through blood vessel endothelium. Circulating cancer cells are those that move within the bloodstream, as opposed to cancer cells present at a fixed location, such as a solid tumor. The transendothelial migration can be either extravasation or intravasation.

A variety of putative agents can be tested in a method of the invention. For example, for particular polypeptide targets that have been identified, the agent can be a neutralizing antibody or active antibody fragment against that target; or it can be an antisense nucleic acid which specifically targets expression of a target gene of interest; or it can be an interference nucleic acid (such as a small interference siRNA, or a short-hairpin shRNA) which specifically targets expression of a target gene of interest; or it can be a micro-RNA which specifically targets expression of a target gene of interest; or it can be protein or peptide anti-metastatic agent or a polynucleotide that expresses the polypeptide.

An agent that inhibits cancer metastasis by decreasing transendothelial migration may function at any of a variety of steps in the metastatic progression. For example, it may result in the delayed appearance of secondary tumors, slowed development of primary or secondary tumors, decreased occurrence of secondary tumors, slowed or decreased severity of secondary effects of disease, among others. In the extreme, complete inhibition is achieved, and is referred to herein as prevention (e.g., virtually complete inhibition, no metastasis if it had not occurred, no further metastasis if there had already been metastasis of a cancer, or virtually complete inhibition of the growth of a primary tumor caused by re-seeding of the tumor by a circulating cancer cell.

A variety of small molecules can also be tested for their ability to act as anti-metastatic agents. Naturally occurring or synthetic (man-made) small molecules can be used. Suitable small molecules, sometimes referred to herein as “compounds,” can be isolated from natural sources or developed synthetically, e.g., by combinatorial chemistry. In general, such molecules are identified from large libraries of natural products or synthetic (or semi-synthetic) extracts or chemical libraries according to methods known in the art. Those skilled in the field of drug discovery and development, for example, will understand that the precise source of test extracts or compounds is not critical to the methods of the invention. Accordingly, virtually any number of chemical extracts or compounds can be used in the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.).

Alternatively, libraries of natural compounds in the form of bacterial, fungal, plant, and animal extracts are commercially available from a number of sources, e.g., Biotics (Sussex, UK), Xenova (Slough, UK), Harbor Branch Oceanographics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In methods of the invention to identify or evaluate one or more putative blood vessel transendothelial migration-affecting agents, the agents can be administered to the test animal by any of a variety of conventional methods. For example, a putative agent can be added systemically, intraperitoneally, orally, by inhalation, or it can be contacted directly with the tumor.

A putative agent can be introduced into a test animal at any suitable time during a method of the invention. For example, the agent can be introduced before the metastatic cancer cell has been introduced (e.g., to identify an agent that prevents metastasis), or together with or after the introduction of the metastatic cancer cell (e.g., to identify an agent for use in the treatment of an existing metastasis, to prevent further metastases.)

The effect of the agent on the level of blood vessel transendothelial migration into our out of the tissue model can be evaluated in a variety of different ways. Evaluation of blood vessel transendothelial migration into the tissue model can be used to evaluate extravasation of cells, such as cancer cells, into tissue, while evaluation of blood vessel transendothelial migration out of a tissue model can be used to evaluate intravasation of cells, such as cancer cells. Evaluation of the level of transendothelial migration can be carried out by, for example, histological inspection of the tissue model. Alternately, or in addition, labels can be used to aid in the identification of migrating cells.

Any of a variety of conventional labels can be used to label a cell (e.g., a circulating cancer cell) to be used in a method of the invention. For example, suitable labels include green fluorescent protein (GFP), red fluorescent protein (RFP), and luciferase, whose use is described in the Examples. Other conventional labels include DsRed, EYFP, ECFP, EVFP and derivatives of EGFP. See also the markers listed at the web site of BD Biosciences (Clontech). When it is desirable to label two different cell populations at the same time (e.g., metastatic cancer cells and cells of a resident tumor, or metastatic cancer cells inoculated at two different (e.g. orthotopic and/or ectopic) sites in a subject), labels which can be easily distinguished can be used. For example, a first cell type can be labeled with a GFP and a second cell type with RPF; or a first cell type can be labeled with firefly luciferase and a second cell type with Renilla luciferase.

Methods of detecting (e.g., quantitating) detectably labeled metastatic cancer cells in the tissue model will be evident to the skilled worker. For example, when a metastatic cancer cell is labeled with a fluorescent marker, it can be detected by examining, with a fluorescent microscope, a tissue sample from the tumor of the subject. When a metastatic cancer cell is labeled with luciferase, the tumor can be examined in the living subject (e.g. in real time) by measuring light emission (bioluminescence) from the marker.

Methods of detection can be readily quantified by non-invasive photon flux emission measurement of luminescence (luciferase), non-invasive imaging of fluorescence, ex-vivo imaging of luminescence, ex-vivo imaging of fluorescence, fluorescence-activated sorting of tumor cells after dissociation of the extracted tumors into a cell suspension, immunohistochemical analysis of marker proteins, to provide quatitative, reproducible assays.

The method of identifying or evaluating a blood vessel transendothelial migration-affecting agent makes use of a test animal including an implanted tissue mimicking model with a composite vascular structure comprising donor pericytes on test animal vasculature. The donor pericytes can be obtained from a different species from the test animal. For example, in some embodiments, the donor pericytes are human pericytes and a non-human test animal is used, to create a humanized test animal that can be used to evaluate agents affecting blood vessel transendothelial migration in humans.

In some embodiments, the agent being identified or evaluated is one that is already known to have an effect on transendothelial migration. For example, the compound may be known to have an effect on the adhesion of cancer cells to blood vessels. In additional embodiments, the agent is known to have an affect on the activity of pericytes. Examples of agents that have an effect on the activity of a pericyte-affecting molecule can be selected from the group consisting of a platelet-derived growth factor (PDGF)-BB/PDGF receptor 13, a membrane type 1-matrix metalloproteinase, heparin sulphate proteoglycans, N-cadherin, Ang1/Tie-2, transforming growth factor β, hepatocyte growth factor, ephrinB2, vascular cell adhesion molecule 1/α4-integrin, CD146, and Sdf-1/CXCL12.

### Method of Identifying or Characterizing a Metastasis-Facilitating Factor

Another aspect of the present invention provides a method of identifying or characterizing a metastasis-facilitating factor. In this aspect, the test animal including an implanted tissue mimicking model is used to identify potential therapeutic targets. Recent evidence indicates that treatments directly targeting pathways involved in metastasis of cancer cells have a significantly greater chance of success. The method includes first administering or inducing circulating tumor cells in a test animal, and then determining if the level of tumor cell migration into the tissue model in the test animal has increased or decreased. In some embodiments, the increase or decrease can be evaluated by comparison with tumor cell migration into the implanted tissue model of a control test animal. The test animal includes an implanted tissue mimicking model with a composite vascular structure comprising donor pericytes on test animal vasculature in which the metastasis-facilitating factor has been silenced.

The metastatic process involves a number of different steps. After creation of the initial tumor mass, the tumor is vascularized with a capillary network from the surrounding host tissue. Intravasation of the capillary network by tumor cells then occurs, resulting in the creation of circulating cancer cells. Thin-wall venule-like lymphatic channels offer very little resistance to penetration by tumor cells and provide the most common pathways for tumor cells entry into the circulation, although in some cases detachment and embolization of small tumor cell aggregates occur. Tumor cells that survive circulation may then arrest in the capillary beds of organs, after which extravasation occurs, through interaction of the circulating tumor cell with the endothelium, and facilitated by pericytes. Proliferation within the organ parenchyma completes the metastatic process. One aspect of the present invention is directed towards identifying metastasis-affecting factors involved in this process. In particular, the method is directed towards identifying factors involved in tumor intravasation or extravasation through blood vessel endothelium that is facilitated by pericytes.

The method includes the step of administering or inducing circulating tumor cells. Administered tumor cells can be selected from the group consisting of circulating tumor cells, tumor stem cells, cell lines derived from the immortalization of circulating tumor cells, micrometastatic tumor cells, cell lines derived from the immortalization of micrometastatic tumor cells, cell lines derived from immortalized tumor cells that had been previously purified from solid tumors, primary tumor cells from solid tumors, a piece of fresh tumor that has been resected from a solid tumor, primary tumor cells, cell lines derived from immortalized cells that had been previously purified from clinical metastasis (i.e. the PC3 cell line) and any combination of any of those. Alternately, circulating tumor cells can be induced by providing a test animal in which cancer is likely or certain to occur, or by exposing the test animal to conditions or genes known to induce cancer. For example, circulating cancer cells can be induced by stably transfecting suitable genes whose expression is correlated with metastasis into a suitable host cell, preferably a tumor cell, in the test animal. Any of the cell types discussed herein as starting cells for the in vivo selection of metastatic cells can be used.

A circulating tumor cell, as used herein, is a cell that, when introduced into a suitable site in a test animal, can circulate through blood vessels but is capable of forming a new cancer site upon extravasation. A variety of types of circulating tumor cells can be used in a method of the invention, including cells from metastatic epithelial cancers, carcinomas, melanoma, leukemia, etc. The tumor cells may be, e.g., from cancers of breast, lung, colon, bladder, prostate, liver, gastrointestinal tract, endometrium, tracheal-bronchial tract, pancreas, liver, uterus, ovary, nasopharynges, prostate, bone or bone marrow, brain, skin or other suitable tissues or organs. In a preferred embodiment, the cancer cells are of human origin.

Suitable tumor cells for use in the method are known to those skilled in the art. These include, for example, HeLa cells, carcinoma and sarcoma cell lines, well established cell lines such as the human lung adenocarcinoma line Anip 973, as well as human breast cancer lines MDA-MB-231, MDA-MB-468 and MDA-MB-435, human prostate cancer lines PC3 and DU-145, human glioblastoma line 324, mouse melanoma B16, and others that have been reported or may become available in the art, including, immortalized cells prepared in the laboratory. In some embodiments, the circulating tumor cells are melanoma, breast, prostate, or other solid tumor cancer cells.

Cancer cells can be administered to the test animal by any of a variety of conventional methods. For example, the cells can be introduced systemically, e.g. by injection into a ventricle of the heart or major artery (e.g., carotid), which allows the cells to circulate in the arterial system. In another embodiment, the cells are introduced into the subject by tail vein injection. Cells introduced via tail vein injection travel into the lung capillaries, where they become trapped.

The method of identifying or characterizing a metastasis-facilitating factor makes use of a test animal including an implanted tissue mimicking model with a composite vascular structure comprising donor pericytes on test animal vasculature. The donor pericytes can be obtained from a different species from the test animal. In some embodiments, the administered cancer and the pericytes are from the same (e.g., human) species. In further embodiments, the donor pericytes are human pericytes and a non-human test animal is used, to create a test animal that can be used to identify or characterize one or more metastasis-facilitating factors affecting blood vessel transendothelial migration in humans.

A metastasis-facilitating factor is an endogenous biochemical factor that facilitates blood vessel transendothelial migration by a cancer cell. For example, a metastasis-facilitating factor would be considered a metastasis-facilitating factor if it increases or decreases the number of cells that migrate through the blood vessel endothelium into an implanted tissue model. Examples of known factors include various peptides and carbohydrates involved in cell signaling and adhesion, such as CD146. Factors that can be identified involve constitutive and induced molecules expressed in either the invading cancer cell or the local/resident pericyte. These include secreted growth factors and their cognate receptors, cell-cell adhesion molecules and cell-ECM interacting molecules.

The method of identifying or characterizing a metastasis-facilitating factor also involves silencing the putative factor in donor human pericytes of the implanted tissue mimicking model. Silencing, as used herein, refers to inhibiting the expression or activity of the factor, but does not require 100% inhibition. The importance of the factor in facilitating metastasis can then be evaluated by determining if silencing the factor decreases or increases the passage of cancer cells through blood vessel endothelium. The change in level of the passage of cancer cells can be identified based on known levels of cancer cell passage, or can be determined by comparison with cancer cell passage in control test animals in which the factor has not been silenced.

Silencing can occur as a result of blocking the activity of the factor using, for example, neutralizing antibodies specific for the factor, or can occur through blocking the expression of the factor using, for example, gene silencing. Gene silencing can occur either during transcription or translation, and typically reduces gene expression by 70% or more. Gene silencing can be achieved using a variety of methods known to those skilled in the art. For example, gene silencing can be carried out using transient transfection, such as that involving antisense oligonucleotides, ribozymes, or RNA interference. Alternately, gene silencing can be carried out by delivery of short hairpin RNA using viral transduction through adeno-associated virus (AAV), adenovirus and lentivirus with the secondary integration of the transgene to the host cell's DNA and subsequent transmission to cell progeny.

The ability to silence genes plays an important role in molecular and cell biology and can be readily applied through transfection. Gene expression can be effectively reduced or eliminated by introducing small noncoding RNA molecules that inhibit RNA translation though a process termed RNA interference (RNAi). RNA molecules that take part in RNAi pathways include: (i) small interfering RNAs (siRNA), short (20-25 base pairs) double-stranded RNAs; and (ii) microRNAs (miRNAs), a separate class of short single-stranded RNAs (20-22 nucleotides). RNAi-based approaches rely on the inherent cellular machinery, shared among several eukaryotic organisms, to inhibit mRNA translation. RNAi pathways play in important role in regulating gene expression and are also believed to provide a mechanism to protect cells from extraneous nucleotide sequences (e.g., viruses and transposons).

RNAi pathways are elicited through cleavage of double-stranded RNAs (dsRNAs) to produce siRNAs, or by processing of noncoding RNAs to produce miRNAs. These separate RNAi pathways rely on cellular machinery such as the ribonuclease protein DICER and the RNA-induced silencing complex (RISC). DICER initiates the RNAi pathway by processing dsRNA to form siRNAs or mature miRNAs. These RNA molecules can bind to complementary sequences of mRNA within the RISC, and the mRNA can be cleaved by the catalytic component, Argonaute, which ultimately prevents translation.

### Animal Model for Identifying Transendothelial Migration-Affecting Factors or Agents

An additional aspect of the invention provides a test animal for identifying blood vessel transendothelial migration-affecting factors or agents. The test animal includes an implanted tissue mimicking model with a composite vascular structure comprising human pericytes on test animal vasculature that can be used to identify blood vessel transendothelial migration-affecting factors or agents. As a result of the presence of the human pericytes in the non-human test animal, the test animal is a xenograft model.

An important feature of the test animal is the presence of one or more implanted tissue mimicking models. The tissue mimicking model, or tissue model, is a three-dimensional tissue model reflective of a biological tissue, particularly an organ such as bone or liver. As used herein, “tissue model” refers to models mimicking the cell state, tissues, and organs of the body. An organ or tissue, as used herein, refers to a differentiated part of an organism which has a specific function. Examples include parts which have specific functions such as mechanical support, respiration, secretion, or digestion. In some embodiments, the tissue model is composed of normal animal tissue obtained from a suitable animal subject, which can be the test animal being used in the method, or a different compatible test animal. In other embodiments, the tissue model is an artificial tissue model such as an organoid, or that has been otherwise engineered using cell culture or inorganic materials.

The form of the tissue model is not limited, and may be, for example, an epithelial tissue model that can be constructed by culturing, for example, surface epithelial cells and glandular epithelial cells; a connective tissue model that can be constructed by culturing, for example, fibroblasts and fat cells; a muscle tissue model that can be constructed by culturing, for example, myoblasts, cardiac muscle cells, and smooth muscle cells; a nerve tissue model that can be constructed by culturing, for example, nerve cells and glial cells; and an organoid model that can be constructed from a combination of cells derived from two or more tissues. The cells used are not limited to normal mature differentiated cells, and may be undifferentiated cells such as embryonic stem cells, somatic stem cells, and induced pluripotent stem cells; focus-derived cells such as cancer cells; or transformants transfected by exogeneous genes.

In some embodiments, the tissue mimicking model is a bone tissue model. Bone tissue models can be made of organic or inorganic scaffold material, and mimic bone tissue, typically for bone tissue engineering purposes. A variety of bone tissue models are known to those skilled in the art. See Sarkar, S, Lee, B, Korean J Intern Med. 2015, 30(3):279-293. For example, bone tissue models can be constructed using bioceramics, glass and glass ceramics, biopolymers, and graphene. In particular, porous ceramic materials are useful as bone tissue models. Dennis J., Caplan A., J Oral Implantol. 1993;19(2):106-15. In some embodiments, the bone tissue model comprises a porous calcium phosphate ceramic cube. Samavedi et al., Acta Biomater. 2013 9(9):8037-45.

The tissue mimicking model is implanted into the test animal in a manner which allows or encourages acceptance of the model by the test animal. In some embodiments, the test animal can be immunosuppressed to encourage acceptance of the tissue mimicking model. In some embodiments, the test animal includes a single tissue model, while in other embodiments a plurality of implanted tissue mimicking models are present in the test animal. Use of a plurality of tissue models in a single test animal can allow tests for a variety of different factors to be carried out using a single test animal, or for other purposes such as verification or determination of the effect of placement location on the blood vessel transendothelial migration by cells.

The tissue mimicking model initially comprises mesenchymal stem cells. Mesenchymal stem cells are characterized by their ability to produce and secrete structural molecules commonly found within the extra cellular matrix (ECM), though it is well established that mesenchymal stem cells also produces an array of different signaling molecules which can function in both the differentiation of cells (as in the case of the developing organ) as well as the maintenance of stemness, as seen in the mesenchymal components of the intestinal stem-cell niche. The expression patterns of mesenchymal stem cells have been shown to be vastly different dependent on the site of origin, though some general markers expressed in multiple mesenchymal lineages are vimentin, fibronectin and various forms of collagen. In contrast to the cells of the epithelial components of a developing organ mesenchyme is marked by a migrating capability.

Mesenchymal stem cells reside as pericytes in the abluminal aspect of blood vessels (i.e., perivascular niche) in all vascularized tissues in the body. Mesenchymal stem cells have the capacity of recognizing sites of injury, where they reassemble as pericytes and exert immunomodulatory and trophic activities locally.

Upon implantation into the test animal, a proportion of the loaded mesenchymal stem cells into the tissue mimicking scaffold differentiate into ECM-producing cells (e.g., osteoblasts in bone), while some migrate to the basement membrane of the nascent blood vessels that form around and inside the implanted tissue mimicking model, readopting their perivascular nature (i.e., as pericytes). The system of blood vessels, including pericytes, that forms around and inside the implanted tissue mimicking model is referred to herein as a composite vascular structure, which serves to provide vascular support for the tissue mimicking model. In some embodiments, the mesenchymal stem cells and the resulting pericytes have been modified such that one or more genes expressing factors that may play a role in transendothelial migration have been silenced.

The following example is included to demonstrate a preferred embodiment of the invention. It should be appreciated by those of skill in the art that the techniques disclosed in the example, which follow represent techniques discovered by the inventor to function well in the practice of the invention, and thus can be considered to constitute a preferred mode for its practice. However, those of skill in the art should, in light of the present disclosure, appreciate that many changes can be made in the specific embodiments which are disclosed and still obtain a like or similar result without departing from the spirit and scope of the invention.

### EXAMPLE

### Example 1

### Mesenchymal Stem Cells Regulate Melanoma Cancer Cells Extravasation to Bone and Liver at Their Perivascular Niche

Skeleton and liver are preferred organs for cancer dissemination in metastatic melanoma negatively impacting quality of life, therapeutic success and overall survival rates. At the target organ, the local microenvironment and cell-to-cell interactions between invading and resident stromal cells constitute critical components during the establishment and progression of metastasis. Mesenchymal Stem Cells (MSCs) possess, in addition to their cell progenitor function, a secretory capacity based on cooperativity with other cell types in injury sites including primary tumors (PT). However, their role at the target organ micro-environment during cancer dissemination is not known. We report that local MSCs, acting aspericytes, regulate the extravasation of melanoma cancer cells (MCC) specifically to murine bone marrow (BM) and liver. Intra-arterially injected wild-type MCC fail to invade those selective organs in a genetic model of perturbed pericyte coverage of the vasculature (PDGF-B^{ret/ret}) similar to CD146-deficient MCC injected into wild type mice. Invading MCC interact with resident MSCs/pericytes at the perivascular space through co-expressed CD146 and Sdf-1/CXCL12-CXCR4 signaling. Implanted engineered bone structures with MSCs/pericytes deficient of either Sdf-1/CXCL12 or CD146 become resistant to invasion by circulating MCC. Collectively, the presence of MSCs/pericytes surrounding the target organ vasculature is required for efficient melanoma metastasis to BM and liver.

### Materials and Methods

PDGF-B^{ret/ret} Mice:

PDGF-B^{ret/ret} transgenic mice (PDGF-B mutant) were kindly provided by Drs. Betsholtz and Genove (Karolinska Institute, Stockholm, Sweden). These transgenic mice (C57B1/6 background) express a mutant PDGF-B that lacks a C-terminal retention motif required to confine this growth factor to the EC compartment, necessary for the recruitment of pericyte progenitors expressing PDGFRB. Armulik et al., Circ Res 2005; 97(6), 512-523. The impaired PDGF-B binding results in defective pericyte recruitment and coverage of microvessels with fewer pericytes and their partial abluminal detachment from the vessel wall. Gerhardt H, Semb H., J Mol Med 2008; 86(2), 135-144. Given that the Pdgfb^{ret} allele is hypo-functional and PDGF-B^{ret/+} mice are indistinguishable from PDGF-B^{+/+}mice, adult (10 week old) Het, WT and PDGF-B mutant littermate mice (n=5 per group) were used in all experiments. Lindblom et al., Genes Dev 2003; 17(15), 1835-1840.

Bioluminescence Imaging (BLI):

BLI was performed after subcutaneous injection of 200 μl of 12.5 mg/ml of luciferin substrate (Biosynth, Cat# L-8220) using a Xenogen IVIS 200 series system. Fifteen minutes after B16F10 cell infusion, an early BLI was performed to evaluate cell distribution throughout the body. Later, images at days 3, 7 and 12 were obtained to evaluate cancer cells engraftment and their temporal progression as growing metastases. To quantify tumor invasion to target organs, BLI signal was analyzed (d12) in terms of photon flux (photons/second/cm²/steradian) and the area covered by signal (cm²c⁻¹) taken at specific locations (extremities and spine after adrenal glands removal) using a pre-defined geometrical shape.

Gene Silencing in B16F10 MCC and hMSC Cells:

CD146 was silenced in B16F10 MCC using a validated shRNA murine sequence cloned in a regular pLKO.1-puro vector, bacterially amplified, sequence verified and delivered as lentiviral transduction particles ready to use (MISSION® RNAi clone ID: NM_023062.1-656s1c1, Sigma Aldrich, St Louis, Mo.). CD146 and Sdf-1/CXCL12 were silenced in BM-derived hMSC using validated human shRNA sequences cloned into an inducible pLKO-puro-IPTG-3xLacO vector [Isopropyl β-D-1-thiogalactopyranoside (IPTG)-dependent transcriptional induction], also delivered as viral particles (MISSION® RNAi clone IDs: CD146: NM_006500.1-1322s1c1; Sdf-1/CXCL12: NM_000609.4-157s21c1, Sigma Aldrich, St Louis, Mo.). The use of an inducible system is intended to avoid the effects of silencing CD146 and Sfd-1/CXCL12 during the formation of both bone and the sinusoidal network inside the ossicles. For the TEM assay, Sdf-1/CXCL12 gene silencing was induced 5 days before the assay.

In vitro Transendothelial Migration Assay (TEM):

A modified Boyden chamber cell migration assay was used to quantitate the invasion potential of B16F10 cancer cells in 2 different conditions, relative to pre-labeled hMSCs with the cationic lipophilic dye Dil for their fluorescence detection: A) When the MSC/pericytes are in close contact with the membrane but silenced for Sdf-1/CXCL12; and B) When the distance between the membrane (acting as an endothelium) and the MSC/pericytes is increased, reminiscent of the PDGF-B^{ret/ret} mutant mice “anatomic” phenotype (in vitro counterpart).

Humanized Heterotopic Bone Formation Assay:

A total of 4.5×10⁶ non-transduced hMSC and hMSC expressing inducible vectors for non-target (NT), CD146 (CD146.shRNA) and Sdf-1/CXCL12 (Sdf-1.shRNA) were vacuum-loaded into sterile porous ceramic cube carriers (hydroxyapatite/tricalcium phosphate 40/60-Zimmer, Warsaw, Ind.) pre-coated with a 100 μg/ml solution of fibronectin. Dennis et al., Biomaterials 1998; 19(15), 1323-1328. The cubes were subcutaneously implanted into immunocompromised mice (CB17-Prkdc SCID) for 8 weeks to form extraskeletal bone structures (ossicles). Every animal (n=8) received 4 cubes, each representing one of the conditions tested. In order to minimize any potential anatomical effect, their relative positions were changed in each animal. After 8 weeks, gene silencing at the protein level in hMSC was accomplished by administering IPTG (12.5 mM) in the drinking water for 7 days. At this time, 1×10⁶ murine B16F10 MCC were intra-arterially injected. BLI images at days 1, 3, 7 and 12 were taken to evaluate MCC engraftment and growth. Two weeks after cell injections, the animals were sacrificed, imaged and the implanted ossicles analyzed by histology.

Modified B16F10 MCC:

B16F10 cells typically colonize target organs within 2 weeks after systemic injection, at which time metastases can be macroscopically observed as black melanin deposits produced by the engrafted cells. B16F10 cells were lentiviral-transduced with a dual-fusion reporter (fluc-mrfp) encoding firefly luciferase and monomeric red fluorescent protein allowing tracking by Bioluminescence (BLI) and Fluorescence imaging respectively. Love et al., J Nucl Med 2007; 48(12), 2011-2020; Lin et al., Translational Medicine 2012; 1(12), 886-897. Stably transduced cells were sorted by FACS based on red fluorescence intensity, and cells with highest signal collected and serially expanded.

Intra-Arterial Injections of Cancer Cells:

All animal experiments were approved by the local IACUC (Case Western Reserve University) and conform to the U.S. Public Health Service Policy on Humane Care and Use of Laboratory Animals (NIH, Department of Health and Human Services). Five×10⁵ B16F10 cells (in 200 μl of sterile PBS) were delivered intra-arterially under general inhaled anesthesia (2% isoflurane) via a carotid catheter advanced towards the aortic arch, thereby bypassing the lungs and avoiding the first passage effect. Lin et al., Molecular therapy 2013; 22 (1), 160-168. All animals were injected at the same rate (over 2 minutes), monitored for breathing rate and rectal temperature and followed until full recovery from anesthesia.

Clinical Assessment:

General evaluation of mice throughout the experiments includes: body weight, general aspect, degree of mobility and ambulation in an open space, and qualitative assessment of bone cancer-related pain by evaluating movement-evoked limb lifting during walking. Vermeirsch et al., Pharmacol Biochem Behav 2004; 79(2), 243-251. The limb lifting assessment, although subjective, can provide information regarding early functional consequences in colonized bones that can be correlated with the degree of cancer cells engraftment evaluated by BLI, as pain appears before any radiological evidence of bone colonization.

Animal Dissections:

A thorough body dissection was performed in order to directly assess invasion by cancer cells. Given that B16F10 melanoma cells actively produce melanin, the degree of tissue colonization can be initially estimated by the presence of deposited pigment.

Micro-Computed Bone Scans (μCT) and Bone Morphometry:

Bone osteolysis was assessed in paraffin fixed whole bone samples by μCT imaging using a GE Healthcare eXplore Locus machine. Multiple micro-tomographic slices obtained with a resolution of 20 μm were then reconstructed in 3D renderings for further analysis. Morphometric parameters [bone volume/total volume (BV/TV), trabecular number (TbN), trabecular thickness (TbTh), trabecular spacing (TbSp), bone mineral content (BMC) and bone mineral density (BMD)] were calculated in processed samples and used as indicators of an underlying bone phenotype.

Histology and Immunohistochemistry:

For histology (H&E staining) and IHC, harvested bones and ossicles were fixed in 4% Paraformaldehyde (PFA) for 48 h, decalcified in a solution of 12.5% EDTA/2.5% PFA-pH 7.5 for 10 days (4° C.), and then paraffin embedded and sectioned (10 μm). IHC in sections was performed after antigen retrieval (Proteinase K at 37° C.), endogenous peroxidase quenching (3% H₂O₂) and primary antibody incubation using the ImmPRESS™ polymerized reporter enzyme staining system (peroxidase micropolymers) with different enzyme substrates (chromogens) allowing multiple labeling (Vector Labs). The following optimized primary antibodies were used: Rabbit monoclonal anti-CD146 (Millipore, Billerica, MA, Cat #04-1147); Mouse monoclonal anti-human CD271 (BD Pharmingen Cat #557194) to specifically identify MSC/pericytes of human origin; Rat monoclonal anti-CD31 (Abcam ab56299, Cambridge, Mass.) to identify endothelial cells.

Gene Silencing in B16F10 MCC and hMSC Cells:

Cultures of hMSC were established as previously described. Haynesworth et al., Bone 1992; 13(1), 81-88. The BM was collected using a procedure reviewed and approved by the University Hospitals of Cleveland Institutional Review Board. Cells were obtained from healthy de-identified adult volunteer donors after signing an informed consent. The use of an inducible system is based on the lactose operator-repressor system that efficiently suppresses target gene expression both in vitro and in vivo after 24 h of IPTG administration to mice in the drinking water (12.5 mM). Wu et al., DNA Cell Biol 1997; 16(1), 17-22. In all cases, 100,000 cells per well (6 well plate) were transduced in 1.5 ml total volume containing Protamine Sulfate (100 μg/mL) as coadjuvant, and viral particles at MOI 5. Selection was performed for 10 days with Puromycin (5 μg/ml) with non-transduced cells serving as selection control. Amplified transduced cells were tested for silencing efficiency by immunocytochemistry in coverslip-cultured cells (CD146) and qPCR in regular cultures (Sdf-1/CXCL12) using a non-target (NT) sequence as control. For the inducible silencing vectors, transgene activation was tested using two different concentrations (200 μM and 1 mM) of IPTG (Life technologies, Carlsbad, Calif.) for 6 days. For qPCR assessments, cells were collected by centrifugation for 5 min at 1200 rpm (110×g) and RNA isolated with TRIzol (Invitrogen) followed by DNasel digestion and purification with the RNeasy mini kit (Qiagen). One μg of high quality total RNA was retrotranscribed with SuperScript III (Invitrogen), and 10 ng of the resulting cDNA was amplified by qPCR in a StepOne Real-time thermocycler (Applied Biosystems) using SYBR-green. Results werenormalized to the endogenous expression of GAPDH and the fold expression calculated with the 2^{-AA}CT method.

In vitro transendothelial migration assay (TEM):

A) When the MSC/pericytes are silenced for Sdf-1/CXCL12: DiI-labeled hMSCs (5×10⁵) expressing either NT_shRNA or Sdf-1/CXCL12_shRNA vectors were treated with IPTG (200 μM) for 5 days to induce gene silencing. They were then cultured at the bottom of an 8 μm pore size polyethylene therephthalate membrane pre-coated with Gelatin (1% for 1 h at 37° C.) and a thin layer of Matrigel (BD BioCoatTM Matrigel™) facing the upper chamber following manufacturer's instructions. As controls, no cells and human papillary dermal fibroblasts were cultured similarly. Forty-eight hours after culturing the coated membrane with the engineered hMSCs, 1×10⁵ B16F10 melanoma cancer cells were seeded in the upper chamber and allowed to migrate through the membrane for additional 48 hours. The upper chamber was then scrapped with a cotton swab to remove unattached cells and the membrane removed from the insert with a scalpel, fixed in 10% Neutral Buffered Formalin and analyzed by bright field and fluorescence microscopy merging both images. B) When the distance between the endothelium and the MSC/pericytes is increased: The TEM was performed comparing the results obtained in A (distance of ˜50 nm) with different distances between the hMSCs and the coated membrane (<1 mm, 1 mm and >1 mm). The hMSCs were plated on the bottom surface of the receiving plate and the B16F10 cancer cells were seeded on the upper chamber coated with Matrigel and Gelatin in an insert with adjustable heights (carrier plate system —Nunc®, Thermo Scientific).

Statistical Comparisons:

BLI data from injected animals (photon flux and area covered by tumors) were pooled individually (extremities and spine of each mouse) and statistical difference between PDGF-B mutant and Het mice calculated using a paired T-test. BLI data from engineered cubes (photon flux) were compared and statistical difference calculated following a one-way ANOVA with Tukey's multiple comparison tests (contrasted to control cubes).

### Results

Skeletal and Liver Melanoma Tumor Burden is Reduced in PDGF-B^{ret/ret} Mice:

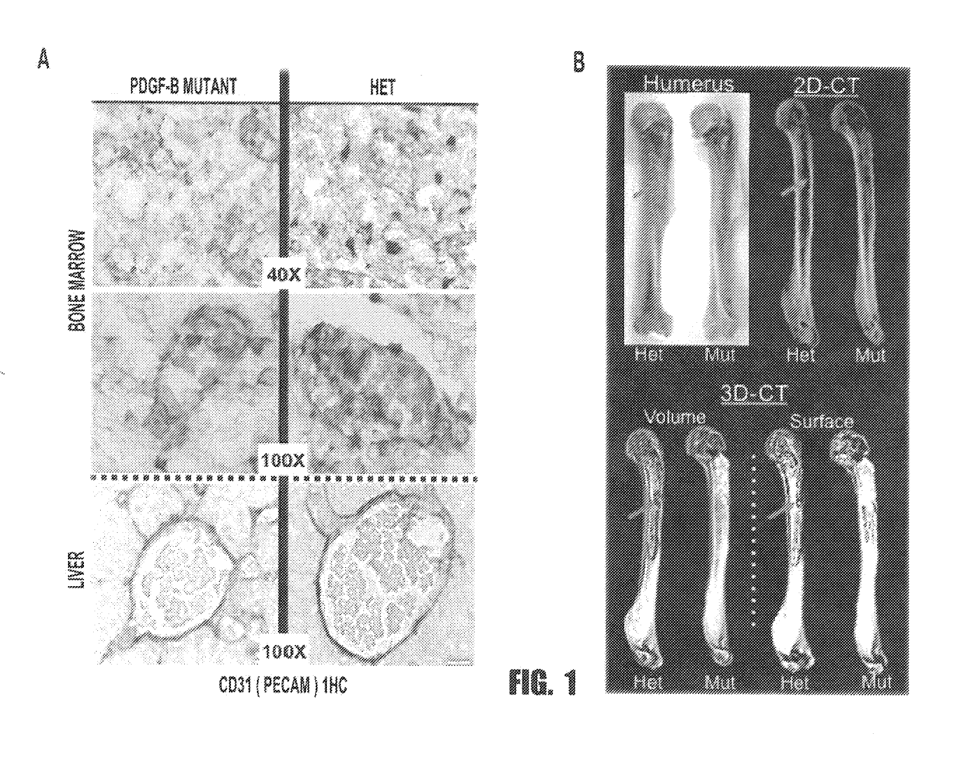

In PDGF-B mutant mice no underlying bone and gross vascular phenotypes (FIG. 1A) were observed. Twelve days after B16F10 cell injection, both heterozygous (Het) and wild type (WT) mice exhibited marked clinical deterioration including severe cachexia (32±3% body weight reduction vs. 20±2% in PDGF-B mutant mice, P<0.05), restricted mobility/ambulation, hunched backs, increased movement-evoked limb lifting and respiratory distress, not observed in PDGF-B mutant mice.

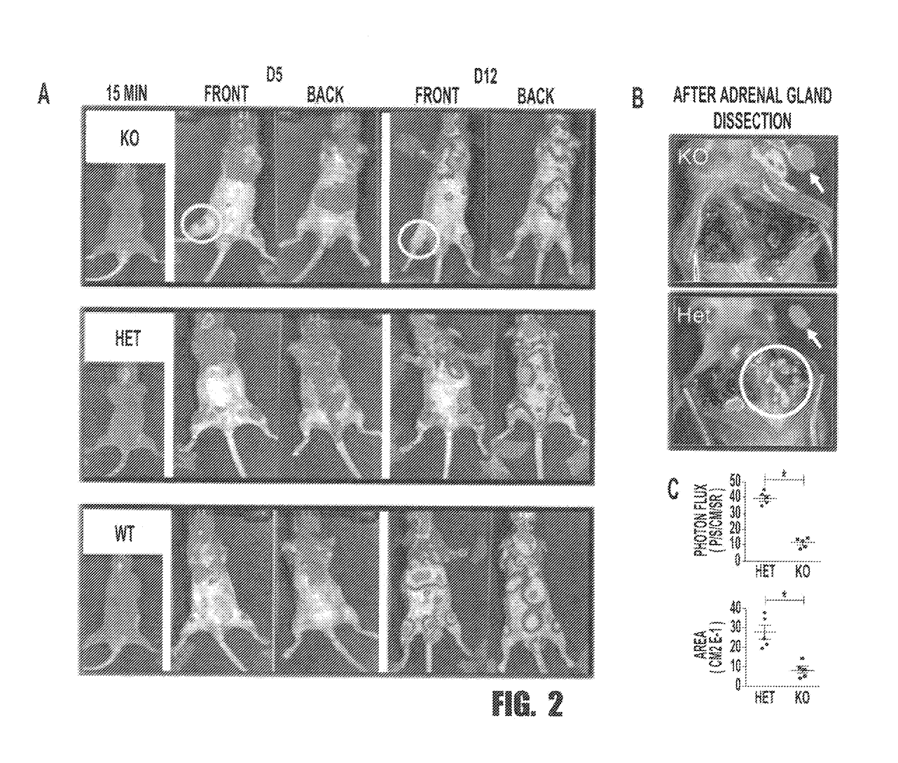

BLI assessment shows that WT and Het mice exhibit an increased skeletal tumor burden as compared with PDGF-B mutants, determined by the number of metastatic foci in the extremities, pelvis and spine, and by their signal quantification (FIG. 2). In PDGF-B mutant mice (n=3) some metastatic foci disappeared in time (FIG. 2A—yellow circles). The extensive compromise of long bones seen in Het controls was linked with functional bone osteolysis, analyzed by two (2D)- and three (3D)-dimensional volumetric micro computed tomography (μCT) reconstruction (FIG. 1B).

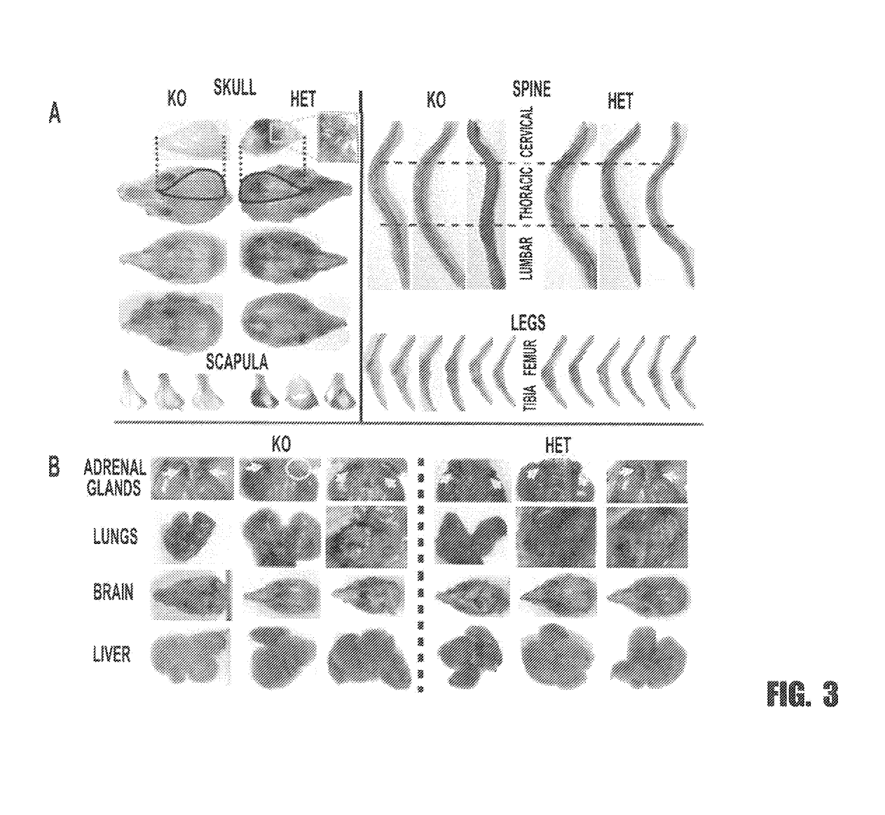

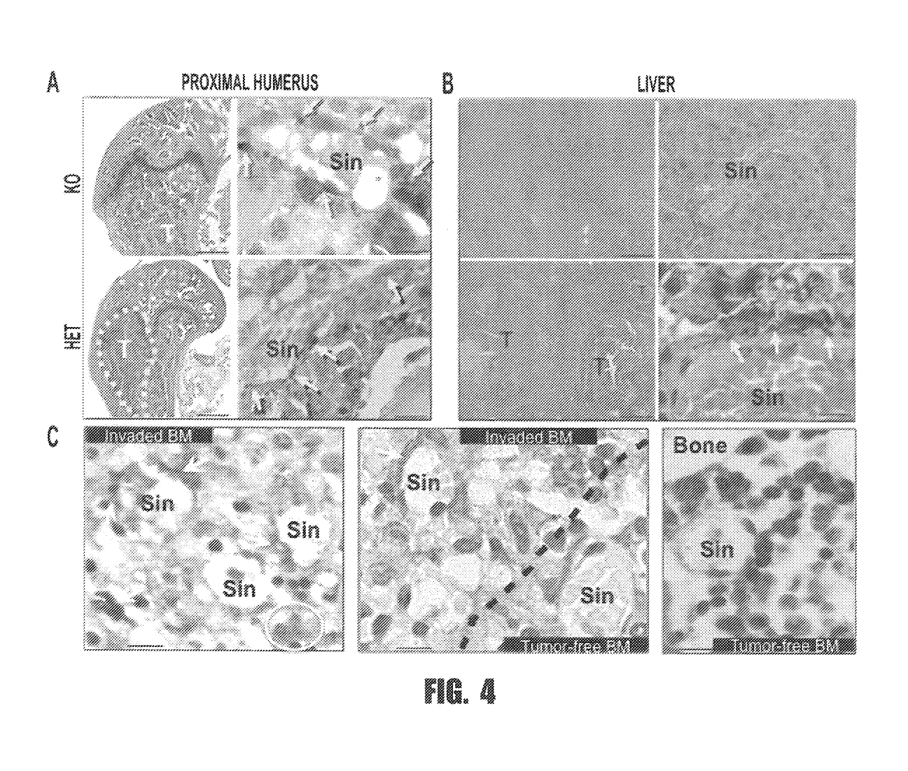

Animal dissections confirmed the overall significant reduction in skeletal invasion in PDGF-B mutant mice compared with Het controls (FIG. 3A), also evident in liver but not in other melanoma target organs including adrenal glands, lungs and brain (FIG. 3B). In addition to the significantly reduced overall macroscopic tumor burden, histological analysis revealed comparable reductions in tumor size in PDGF B mutant mice (FIG. 4A). Similarly, no metastatic foci were observed histologically in PDGF-B mutant livers compared with the multifocal invasion in WT and Het mice (FIG. 4B).

MCC Establish a Perivascular Niche During Engraftment/Invasion in the Skeleton and Liver where They Interact with Resident Murine MSC/Pericytes:

High power microscopy analysis of all histological specimens across genotypes revealed the absence of micrometastatic foci in regions where invasion was not visible macroscopically. Melanin producing cancer cells were observed abluminally with respect to BM and liver sinusoids, adopting a perivascular location. In WT and Het mice, the invaded MCC colonized the BM parenchyma where they appear to reside in physical association with CD146-expressing resident MSC/pericytes (FIG. 4C). This is in contrast to PDGF-B mutant-derived samples where no further advancement of invading cells was observed beyond the perivascular space (FIG. 4A, high magnification).

This physical association and the constitutive expression of CD146 by both MCC and MSC/pericytes prompted us to evaluate CD146 as a potential mechanism for an intercellular adhesion between them at the perivascular space generated during extravasation.

CD146 Silencing in MCC Impairs Their Ability to Extravasate to Skeleton and Liver:

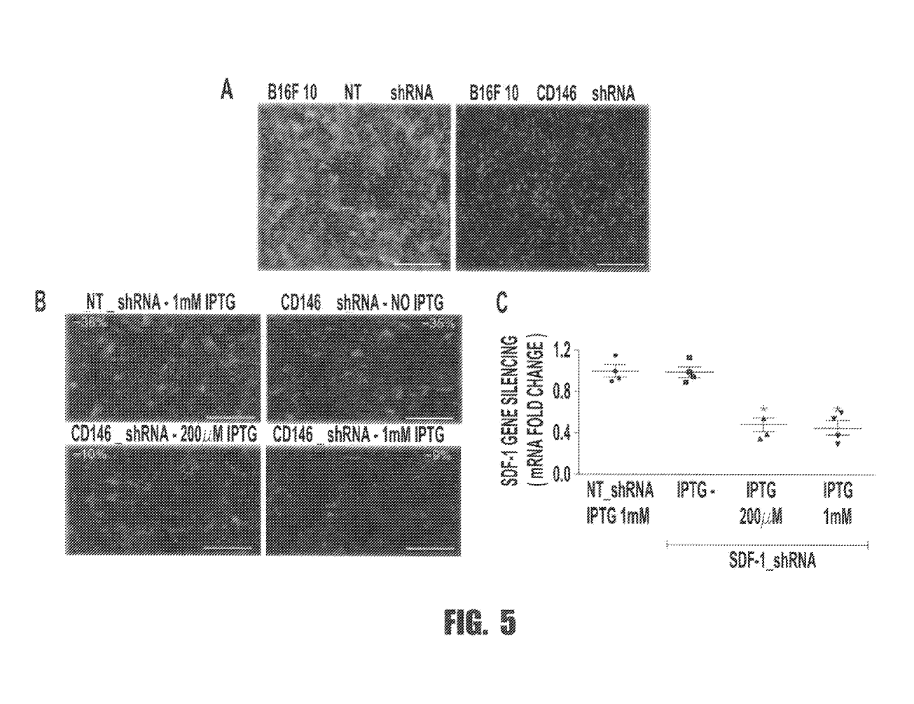

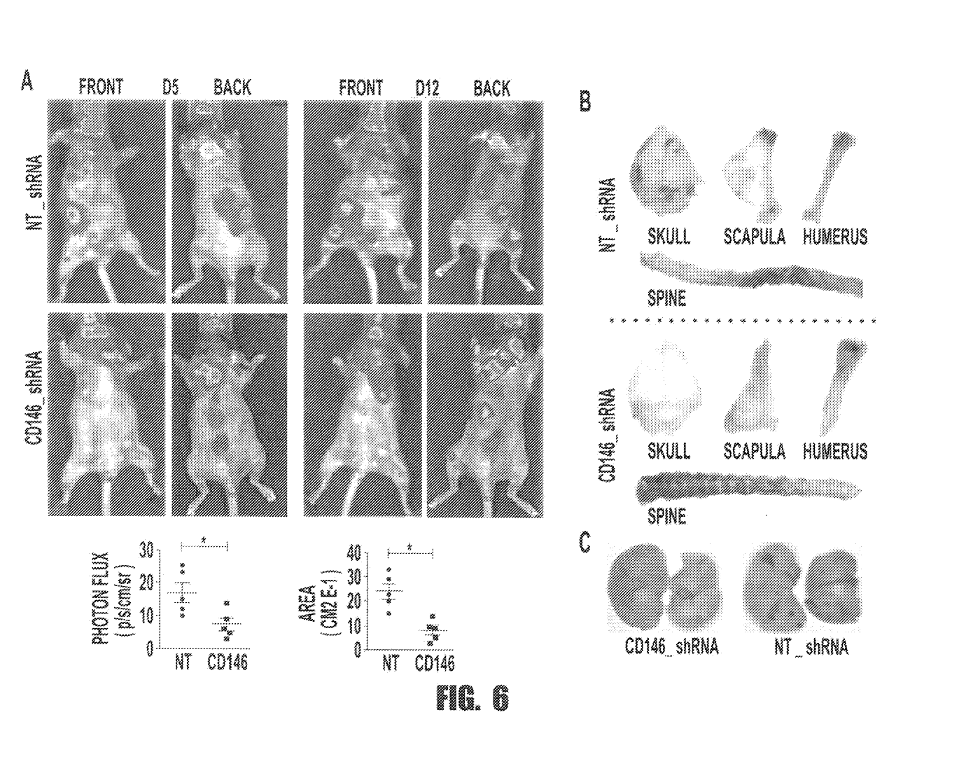

We first assessed the role of CD146 from the invading cell perspective. A high CD146 silencing efficiency (˜90%) was obtained after lentiviral transduction of B16F10 cells with the constitutive CD146_shRNA vector as compared with the NT (non-targeting)_shRNA control (FIG. 5A). These engineered cells were then intra-arterially injected into WT mice (n=6) Animals that received control cells (NT_shRNA; n=3) exhibited a more rapid and dramatic clinical deterioration compared with animals that received cells with silenced CD146, including a more pronounced weight loss (34±3% vs. 18±2%, P<0.05), ambulation difficulties, restricted limb movements and hunched backs. Similar to PDGF-B mutant mice, invasion was compromised selectively to skeleton and liver in mice that received CD146-silenced B16F10 cells compared to control cells (FIG. 6).

CD146 Silencing in BM-Derived hMSC/Pericytes Impairs MCC Invasion to Humanized Extraskeletal Bone Structures in mice:

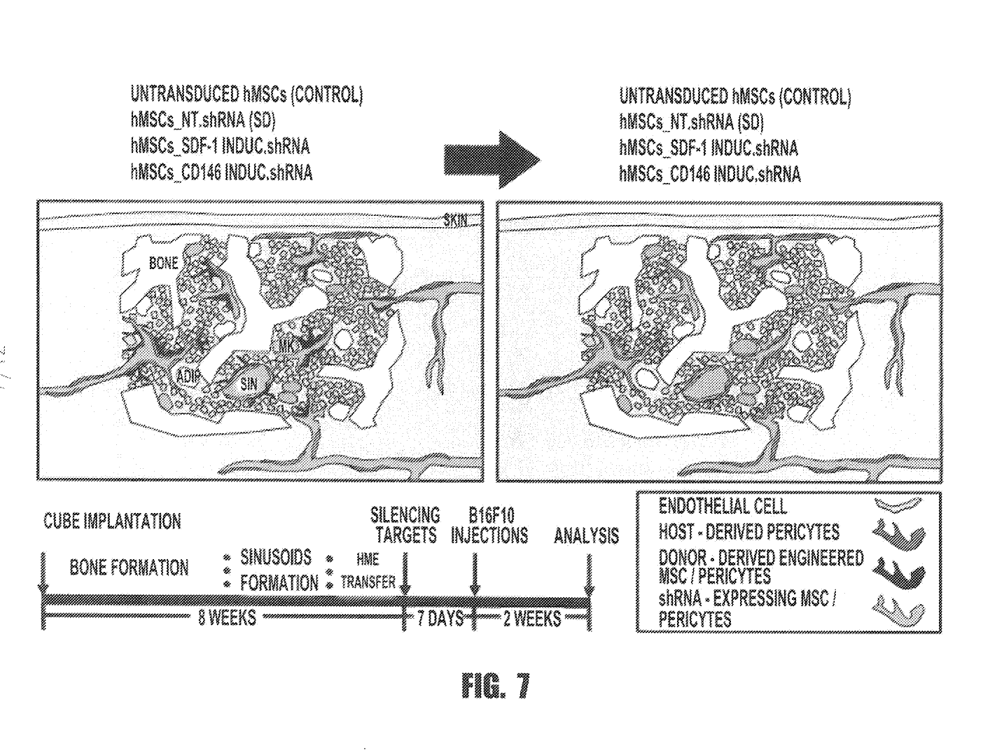

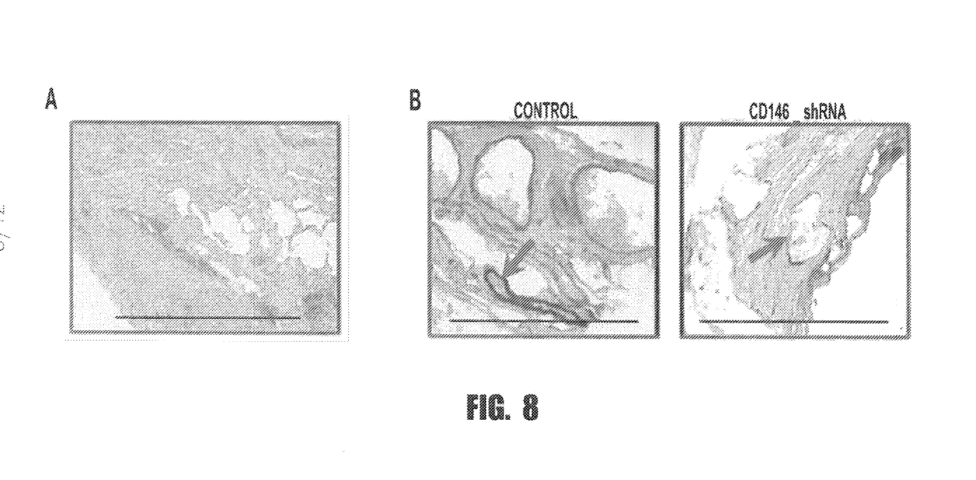

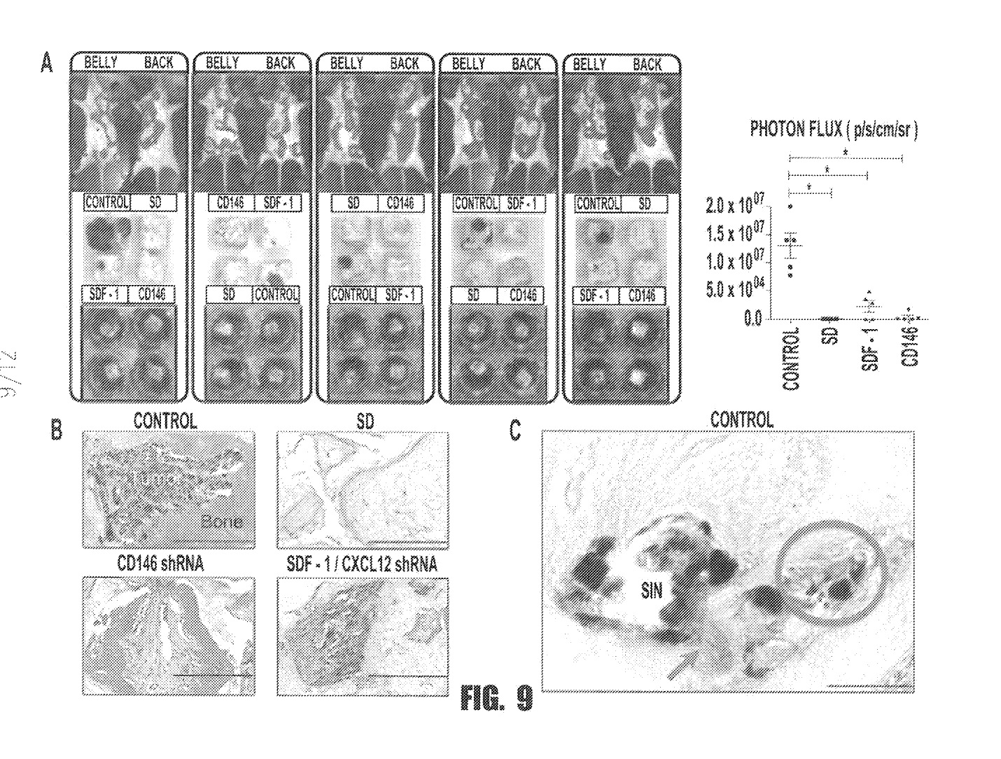

As CD146 has been shown to exhibit homotypic interactions, we next evaluated the role of CD146 silencing now from the resident MSC/pericyte perspective. The two concentrations of IPTG tested resulted in a comparable silencing efficiency of CD146 in hMSC (˜75%) as assessed by immunolocalization of the protein in cultured cells (FIG. 5B). Engineered cells were used to create osteogenic ceramic cubes (depicted in FIG. 7), which after implantation in immunocompromised mice generated humanized extraskeletal bones (ossicles) that fully recapitulate the native bone structure including the formation of blood vessels (sinusoids) and sequentially functional hematopoietic tissue. Within these humanized osseous structures, donor-derived hMSC form the bone tissue and assemble as pericytes during the formation of vascular structures, as shown by Sacchetti et al, and confirmed by our observations with immunolocalization of the hMSC marker CD271 using a specific anti-human antibody (FIG. 8A). Sacchetti et al., Cell 2007; 131(2), 324-336. This humanized assay allowed us to circumvent the use of a CD146 KO mouse to monitor the effects of CD146 deficiency on the ability of resident perivascular cells to drive melanoma cell extravasation into the BM. As a control, untransduced WT hMSC (control) and a subset of hMSC with pathologically slow dividing activity after being transduced with the NT_shRNA vector and passaged several times (SD) were used. These two controls represent normal (control) and an aberrant (SD) formation of the ossicles and all their structural components of which the SD cells reflected the reduced osteogenic capacity of hMSC after serial passaging. Eight weeks after subcutaneous ossicle implantation, IPTG (12.5 mM for 7 days) was administered through the drinking water to induce the in vivo gene silencing within these bone structures, evidenced by immunolocalization of CD146 in sections (FIG. 8B), and consistent with the in vitro silencing. Intra-arterially injected MCC were found to invade the skeleton and the control hMSC-made ossicles as evidenced by the intense BLI signals that were observed in all structures (5/5) (FIG. 9A). In contrast, ossicles made with hMSC that lacked CD146 generated a dramatic reduction in the invasion of MCC, detected in only one structure (1/5) with a faint signal. As expected, ossicles made with slow dividing hMSC (SD) were not invaded by MCC, as these structures lacked substantial bone formation, vascular structures and hematopoietic tissue observed in a histological analysis (FIG. 9B). Finally, like the situation in the murine BM, invading MCC were found in humanized ossicles in close spatial relationship with resident hMSC/pericytes identified by immunolocalization of CD146 (FIG. 9C).

Sdf-1/CXCL12 Silencing in BM-Derived hMSC/Pericytes is Required for MCC to Invade Humanized Extraskeletal Bone Structures and to Migrate in vitro:

Sdf-1/CXCL12 has been described as a potent attractant to CXCR4-expressing cells, including MCC. Bartolome et al., Cancer Res 2004; 64(7), 2534-2543. Its expression in the BM has been demonstrated to come primarily from perivascular cells (i.e., MSC/pericytes). Ding L, Morrison S J, Nature 2013; 495(7440), 231-235. Therefore, we assessed its potential contribution during extravasation of MCC into bone, through it silencing in resident MSC/pericytes using the humanized extraskeletal bone formation assay. The two concentrations of IPTG tested to induce Sdf-1/CCXCL12 gene silencing generated a comparable ˜55% reduction relative to untreated parental hMSC, or to NT_shRNA-transduced hMSC treated with IPTG (FIG. 5C). Sdf-1/CXCL12 silencing in hMSC/pericytes showed a significant reduction both in the number of ossicles invaded (2/5) and the intensity of the BLI signal obtained (FIG. 9A), as well as on the size of the secondary tumors where present (FIG. 9B).

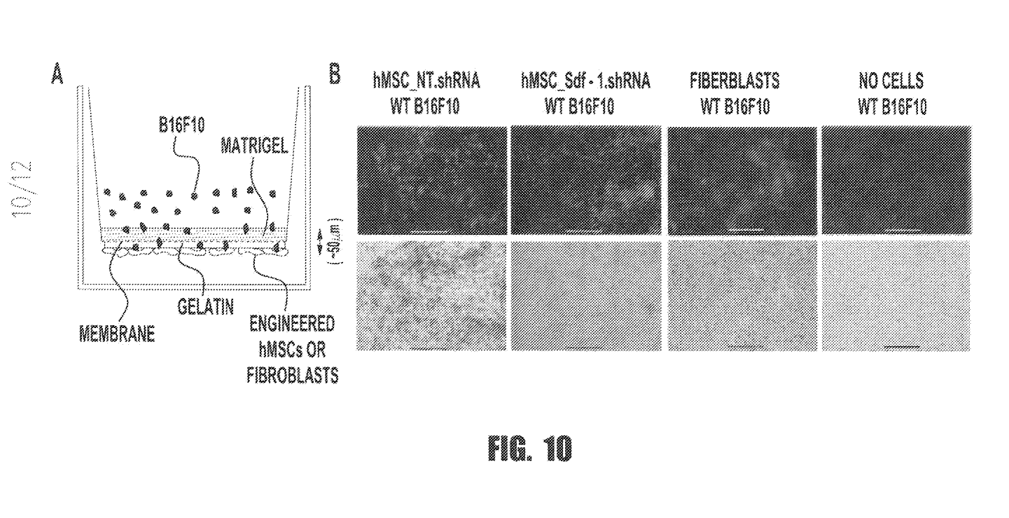

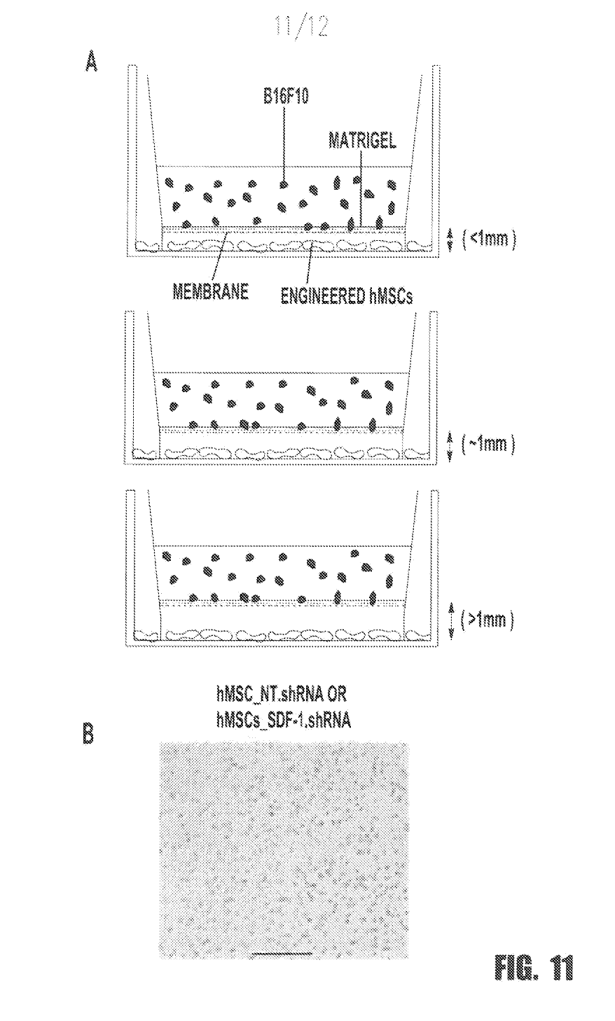

In an in vitro modified transendothelial migration (TEM) assay (FIG. 10A), fluorescence microscopy revealed DiI pre-labeled hMSC that were seeded at the bottom of an 8 μm pore membrane forming colony-like structures (FIG. 10B—top row). Bright-field microscopy showed the interaction of B16F10 cells (after migrating from the upper chamber) with parental hMSC (i.e., NT_shRNA control cells) in that same plane, an event that was noticeably absent in their Sdf-1/CXCL12-deficient counterparts and when skin fibroblasts and no cells were used as controls (FIG. 10B—bottom row). Similarly, an increased distance between the MCC and hMSC prevented their migration throughout the membrane regardless of whether the hMSC were silenced or not for Sdf-1/CXCL12 (FIG. 11).

### Discussion

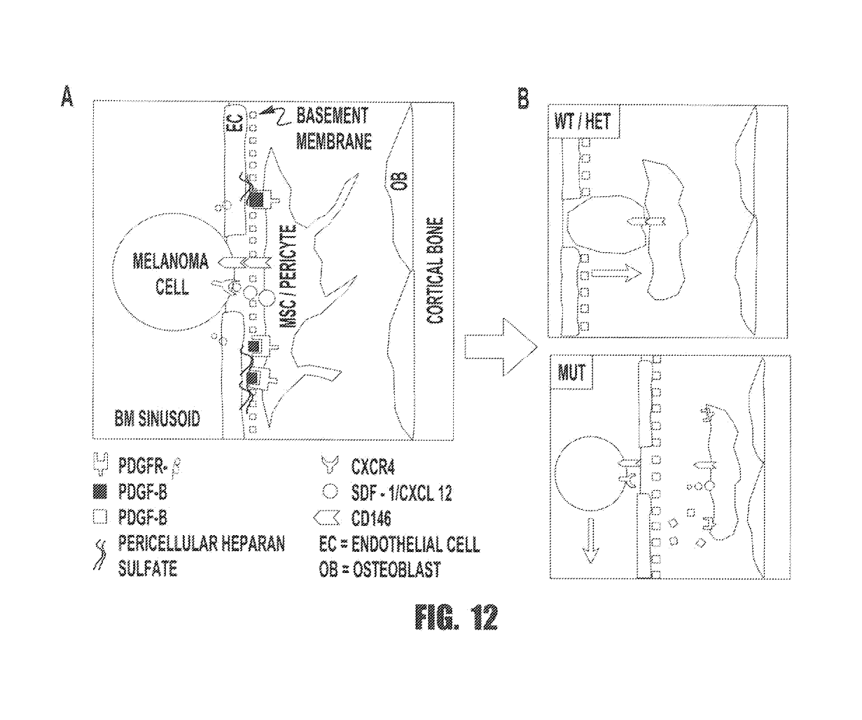

Through the use of complementary in vitro and in vivo approaches, including a humanized assay of bone metastases in mice, we documented details of the function of MSC/pericytes in mediating the extravasation and the initial metastatic seeding of MCC at the BM and liver microvasculature. Mechanistically, we describe the participation of the cell surface molecule CD146 and the chemokine Sdf-1/CXCL12 as critical determinants of the molecular events occurring during the dissemination process resulting in the physical association between the invading cancer cell and the MSC/pericyte at the target organ microenvironment (i.e., perivascular space). Importantly, genetic ablation of abluminal positioning of pericytes alleviates these untoward events, and as such, we propose that circulating MCC follow an Sdf-1/CXCL12 gradient that facilitates their access to endothelial fenestrations and resident MSC/pericytes. These MSC/pericytes specifically associate with CD146-positive MCC and promote their extravasation into the target organ parenchyma (depicted in FIG. 12).

The concept of a physical association between invading cancer cells and stromal cells in the BM has been previously reported as critical for the progression and fate of metastatic tumors. Yoneda T, Hiraga T., Biochem Biophys Res Commun 2005; 328(3), 679-687. In addition, the participation of MSC during the endothelial transmigration of low metastatic breast cancer cells has been suggested using in vitro models. Nevertheless, our study constitutes the first direct in vivo evidence of the participation of MSC as pericytes during the process of melanoma dissemination, as well as the description of the precise location (i.e., perivascular space) and the molecular players involved in the cell-to-cell association that lead to the establishment of distant metastases. We observed a close proximity between invading MCC and resident MSC/pericytes at both the sinusoidal perivascular space and the tissue parenchyma. These observations further suggest the physical interaction between those two cell types during the extravasation of cancer cells at the target organ and establishes a previously underappreciated sentinel role of MSC (as pericytes) during the process of cancer cell invasion.

MSC have been historically seen as precursors of mesenchymal tissues including bone, cartilage, fat and muscle. Nevertheless, our data supports the recent proposition that MSC reside in a perivascular niche and that they arise from perivascular cells, while suggesting that they participate in parallel homeostatic functions exerted at their strategic abluminal location including cancer invasion to target organs. Indeed, the function of pericytes during cancer dissemination is not limited solely to their known effect on vascular stability (Armulik et al., Circ Res 2005; 97(6), 512-523), but instead indicates a more active role during the process of cancer cell extravasation. In fact, pericytes have been described as gatekeepers of tumor metastasis, since their absence promotes cancer cell dissemination to target tissues from primary tumors in mice. Xian et al., J Clin Invest 2006; 116(3), 642-651. Using the same genetic model of pericyte disturbed coverage reported by Xian et al (PDGF-Bret/ret mice), we now present evidence that invasion of circulating MCC is significantly and selectively reduced in the skeleton and liver, but remains surprisingly intact at other target organs (e.g., brain, adrenal glands and lungs). These apparent discrepancies may be explained by the following arguments. First, Xian et al focused on the process of cancer cells intravasation from a primary tumor (insulinoma) where an “abnormal” tumor forming vasculature is further destabilized in the mutant mice, thereby increasing the number of tumor cells capable of escaping into the vasculature. This increased number of circulating cancer cells might account for the distant colonization observed in a fraction of the animals. In stark contrast, we focused on the process of extravasation of a predetermined, fixed number of intra-arterially injected MCC from a vessel network at the target organ impacted only by the expression of the transgene. Second, Xian et al studied an insulinoma primary tumor, which makes comparisons of distal invasion difficult to assess because insulinoma cells typically do not invade the BM and liver as proficiently as MCC. Third, it has been proposed that primary tumors secrete factors that “prepare” the target organ for future invasion, a process known as pre-metastatic niche formation. Psaila B, Lyden D., Nat Rev Cancer 2009; 9(4), 285-293. In contrast to the model of Xian et al, our study bypassed the formation of a primary tumor, thus preventing this contributing phenomenon. Finally, Xian et al described both lymphatic and hematogenous pathways as contributors of distant dissemination. Our model does not involve a lymphatic-mediated mechanism as we inject the cancer cells intra-arterially. In addition, truly lymphatic-dependent bone metastases are still controversial. Edwards et al., Human pathology 2008; 39(1), 49-55.

The impaired melanoma invasion to BM and liver observed in the mutant mice suggests the presence of unique mechanistic traits within these two organ sites that are not shared with other target organs. These traits may only become evident in the absence of perisinusoidal cells (MSC/pericytes in the BM and stellate cells in the liver), conferring them “resistance” to metastasis from melanoma. One potential explanation for this phenomenon takes into consideration that BM and liver have hematopoietic capabilities and may share a similar HSC niche, which is known to be targeted by osteotropic cancer cells for the establishment of footholds during skeletal metastasis. Shiozawa et al., The Journal of clinical investigation, 2011; 121(4), 1298-1312. Recently, hepatic stellate cells were observed to represent pericyte-like, CD146-positive liver resident MSC that associate with sinusoidal ECs in close contact with hematopoietic progenitor cells. Kordes et al., Cellular physiology and biochemistry: international journal of experimental cellular physiology, biochemistry, and pharmacology 2013; 31(2-3), 290-304. This situation is reminiscent of the molecular mechanisms we establish herein for MSC/pericytes in the BM. In addition, hepatic stellate cells share a similar pattern of recruitment and alignment around vessel walls as other pericyte cells, and as such, they are highly dependent on the secretion of PDGF-B by ECs. Lee et al., Hepatology (Baltimore, Md.) 2007; 45(3), 817-825. Thus, both of these target organs share similar structural patterns surrounding sinusoids (i.e., stem cell niche), which apparently determine the invasive capacity of circulating MCC.

There are a number of reported mechanisms involved in the regulation of metastatic MCC to target organs, including the interaction between MCC and ECs, and platelets, all of which are mediated through adhesion molecules such as integrins and selectins. Fritzsche et al., Thromb Haemost 2008; 100(6), 1166-1175; Nash et al., The lancet oncology 2002; 3(7), 425-430. CD146 (MCAM, MUC18) is a transmembrane glycoprotein that belongs to the immunoglobulin superfamily. Its aberrant expression regulates the tumorigenicity of MCC (Schlagbauer-Wadl et al., Int J Cancer 1999; 81(6), 951-955), and is associated with the aggressiveness, poor prognosis and metastatic potential of human melanoma when compared to normal skin. Talantov et al., Clin Cancer Res 2005; 11 (20), 7234-7242. It has been reported as part of a “metastases aggressiveness gene expression signature” present in melanoma metastases in human patients, establishing an essential role of the tumor cell-target organ microenvironment interactions during metastasis, as we demonstrated here between circulating MCC and abluminal MSC/pericytes. Xu et al., Molecular Cancer Research 2008; 6(5), 760-769. In addition, CD146 has been shown to affect melanoma cell extravasation to lung during dissemination, by interacting with ECs and mediating VEGF-induced vessel permeability. Jouve et al., International journal of cancer Journal international du cancer 2015; 137(1), 50-60.

Homotypic interactions between CD146 molecules have been proposed as a mechanism for increasing cohesive cell-cell interactions between similar and different cell types including MCC (e.g., B16 MCC and B-1 lymphocytes). Johnson et al., Int J Cancer 1997; 73(5), 769-774. Our results demonstrate the necessity of CD146 to be expressed in both the invading MCC and resident MSC/pericytes for efficient extravasation. This notion is supported by the significant reduction in invasion observed in both WT mice injected with CD146-deficient MCC and the ossicles made with CD146-silenced hMSC/pericytes. This novel humanized assay approach, to the best of our knowledge, represents the first time functional extraskeletal bone structures are created with engineered human cellular precursors (i.e., MSC) to manipulate the target organ microenvironment and to test the role of specific molecular targets during invasion by MCC. This approach provides information that corroborates the human findings in terms of CD146 relevance directly using humanized tissues in rodents. In addition, it not only permits direct side-by-side comparisons between the different genetic backgrounds obtained with engineered ossicles implanted in the same animal, but also to contrast the invasion rates with the animal skeleton used as an internal control. Another advantage with this approach is that we were able to rule out a potential contribution of ECs-derived CD146 during cancer cell extravasation to bone, as these are unmodified host-derived cells, and it has been reported that BM ECs do not express CD146. Sacchetti et al., Cell 2007; 131(2), 324-336. This CD146 cell source discrimination permits to establish a mechanistic difference in melanoma extravasation between BM and other organs (e.g., lung). Unlike the lung, where EC-derived CD146 is indispensable, MSC/pericyte in the BM supply CD146 during extravasation.

We then hypothesize that the absence of CD146 impacts MCC extravasation by at least two parallel mechanisms. First, CD146-deficiency prevents the formation of cellular complexes between invading MCC and resident MSC/pericytes at the perivascular space, thereby limiting further penetration to the parenchyma. And second, loss of CD146 elicits cell detachment and subsequent apoptosis (anoikis), a process initiated by the loss of cell interactions with the extracellular matrix and/or neighboring cells. Kim et al., International journal of cell biology 2012; 2012, 1-11. CD146 has been shown to promote cancer cell resistance against anoikis by facilitating cell-cell interactions, which may upregulate anti-apoptotic mechanisms, a mechanism disrupted when CD146 is not present in either invading MCC and/or the resident MSC/pericytes. Wai et al., International journal of cell biology 2012; 2012, 340296. This would explain the impaired extravasation observed in the in vivo model of deficient vascular pericyte coverage where the absence of CD146-expressing MSC/pericytes fails to provide anoikis resistance and the generation of the translocating cellular complex. In addition, this mechanism would also explain the disappearance of the signal obtained with MCC over time in some mutant mice.

Using a similar in vivo approach we established the role of Sdf-1/CXCL12 secreted by resident MSC/pericytes during extravasation, observing a drastic reduction of melanoma cell invasion into humanized ossicles silenced for Sdf-1/CXCL12 in MSC/pericytes. Sdf-1/CXCL12 is a pro-survival chemokine that serves as a potent chemoattractant for circulating hematopoietic precursors (Mendez-Ferrer et al., Nature 2008; 452(7186), 442-447), as well as various bone-metastasizing cancers, including melanoma, expressing its cognate receptor CXCR4. In the BM, various cell types including perivascular, endothelial and osteoblastic cells express Sdf-1/CXCL12. Dar et al. Nature immunology 2005; 6(10), 1038-1046. However, it has been demonstrated that perisinusoidal cells (i.e., MSC/pericytes) referred by Sugiyama et al as CXCL12-abundant reticular cells (CAR) constitute the main source of this factor, compared with a reduced expression and secretion by ECs. Sugiyama et al., Immunity 2006; 25(6), 977-988. These results with Sdf-1/CXCL12 emphasize the striking similarities in the entry (and potentially exit) to the BM between circulating osteotropic MCC and HSCs, further supporting the findings with prostate cancer cells by Shiozawa et al. Shiozawa et al., The Journal of clinical investigation, 2011; 121(4), 1298-1312. In addition, the host-derived low secretion of ECs in the ossicles vasculature might explain the reduced (not absent) MCC invasion to structures made with silenced Sdf-1/CXCL12.

Taken together, the pivotal role of MSC/pericytes at their perivascular niche regulating the extravasation of circulating MCC to bone and liver further expands our knowledge about novel functions of these adult stem cells. In parallel, the identified molecular mechanisms involving intercellular adhesion molecules (i.e., CD146) and secreted chemokines (i.e., Sdf-1/CXCL12) strengthens the concept of cellular cooperativity reported at sites of injury and primary tumors and now expanded to target organs during cancer dissemination. Finally, these mechanisms may serve as a platform for the development of novel therapies aimed at controlling the establishment and progression of skeletal and liver metastasis from melanoma by targeting MSC/pericytes.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

What is claimed is:

1. A method of identifying or evaluating a blood vessel transendothelial migration-affecting agent, comprising administering the agent to a test animal comprising an implanted tissue mimicking model with a composite vascular structure comprising donor pericytes on test animal vasculature, and determining the effect of the agent on the level of blood vessel transendothelial migration into or out of the tissue model.

2. The method of claim 1, wherein the blood vessel transendothelial migration is the facilitated transport of circulating cancer cells, and the blood vessel transendothelial migration-affecting agent is an antimetastatic agent.

3. The method of claim 1, wherein the tissue mimicking model is a bone tissue model.

4. The method of claim 1, wherein the tissue mimicking model comprises a porous calcium phosphate ceramic cube.

5. The method of claim 3, wherein the blood vessel transendothelial migration is the facilitated extravasation of circulating melanoma cancer cells into the bone tissue model.

6. The method of claim 1, wherein the effect of the agent on facilitated blood vessel extravasation is determined.

7. The method of claim 1, wherein the effect of the agent on facilitated blood vessel intravasation is determined.

8. The method of claim 1, wherein the donor pericytes are human pericytes.

9. The method of claim 1, wherein the blood-vessel transendothelial migration-affecting agent has an effect on the activity of a pericyte-affecting molecule selected from the list consisting of a platelet-derived growth factor (PDGF)-BB/PDGF receptor β, a membrane type 1-matrix metalloproteinase, heparin sulphate proteoglycans, N-cadherin, Ang1/Tie-2, transforming growth factor β, hepatocyte growth factor, ephrinB2, vascular cell adhesion molecule 1/α4-integrin, CD146, and Sdf-1/CXCL12.

10. The method of claim 1, wherein the blood-vessel transendothelial migration-affecting agent has an effect on the activity of CD146 or Sdf-1/CXCL12.

11. A method of identifying or characterizing a metastasis-facilitating factor, comprising administering or inducing circulating tumor cells in a test animal comprising an implanted tissue mimicking model with a composite vascular structure comprising donor pericytes on test animal vasculature in which the metastasis-facilitating factor has been silenced, and determining if the level of tumor cell migration into the tissue model has increased or decreased.

12. The method of claim 11, wherein the tissue mimicking model is a bone tissue model.

13. The method of claim 11, wherein the tissue mimicking model comprises a porous calcium phosphate ceramic cube.

14. The method of claim 11, wherein the donor pericytes are human pericytes.

15. The method of claim 11, wherein the circulating tumor cells are melanoma, breast, prostate, or other solid tumor cancer cells.

16. The method of claim 11, wherein the metastasis-facilitating factor has been silenced by transfection.

17. A test animal for identifying blood vessel transendothelial migration-affecting factors or agents, comprising a test animal comprising an implanted tissue mimicking model with a composite vascular structure comprising human pericytes on test animal vasculature.

18. The test animal of claim 17, wherein the tissue mimicking model is a bone tissue model.

19. The test animal of claim 18, wherein the bone tissue model comprises a porous calcium phosphate ceramic cube.

20. The test animal of claim 17, wherein a plurality of implanted tissue mimicking models are present in the test animal.

21. The test animal of claim 17, wherein a blood vessel transendothelial migration-affecting factor in the human pericytes has been silenced.

22. The test animal of claim 21, wherein the transendothelial migration-affecting factor has been silenced by transfection.
